(19) United States
(12) Patent Application Publication
(10) Pub. No.: US 2016/0296560 A1
(43) Pub. Date: Oct. 13, 2016

(54) A METHOD OF PRODUCING EXOSOMES

(71) Applicant: XOSOMA PTY LTD, Carnegie, Victoria (AU)

(72) Inventor: Bill PASPALIARIS, Carnegie (AU)

(21) Appl. No.: 15/026,858

(22) PCT Filed: Oct. 2, 2014
(86) PCT No.: PCT/AU2014/000953

§ 371 Date: Apr. 1, 2016

(30) Foreign Application Priority Data
Oct. 2, 2013 (AU) 2013903805

(51) Int. Cl. A61K 35/15; G01N 33/50; C12N 15/88; C12N 5/0786

(52) U.S. Cl. CPC A61K 35/15; C12N 5/0645; G01N 33/50; C12N 15/88; A61K 2035/124

(57) ABSTRACT

The present application relates to methods of producing exosomes or extracts thereof for use in the treatment of diseases or disorders. In particular, the present invention relates to a method of producing exosomes or extracts thereof comprising the steps of: (a) exposing a population of isolated mammalian cells to light between 500 nm to 820 nm for sufficient time to enable said cells to produce and excrete said exosomes; and (b) separating said exosomes from other cellular components based on molecular weight, size, shape, composition or biological activity.

### FIELD

The present application provides methods of producing exosomes or extracts thereof for use in the treatment of diseases or disorders. In particular, the present invention provides methods of producing exosomes or extracts thereof by exposing a population of isolated mammalian cells to light between 500 nm to 820 nm for sufficient time to enable said cells to excrete said exosomes.

### BACKGROUND

Exosomes are naturally occurring, preformed, membrane-covered vesicles of 30-150 nm of endocytic origin that are secreted by most cell types in vitro and released in the extracellular milieu following fusion of the vesicular body and plasma membranes (Johnstone (1992), Biochem Cell Biol., 70(304): 179-190; Denzer et al., (2000), J Cell Sci., 19: 3365-3374; Thery et al., (2002), Nat Rev Immunol., 2(8):569-579). They have been identified in vivo in all body fluids including amniotic fluid, urine, and blood (Simpson et al., (2008), Proteomics, 8: 4083-4099). Exosomes bear surface receptors/ligands of the original cells and have the potential to selectively interact with specific target cells (Rana et al., (2012), Int J Biochem Cell Biol., 44: 1574-1584). In addition to numerous lipids and proteins, exosomes also contain mRNAs and miRNAs (Hergenreider et. al., (2012), Nat Cell Biol., 14: 249-256; Skog et al., (2008), Nat Cell Biol., 10: 1470-1476; Valadi et. al., (2007), Nat Cell Biol., 9: 654-659; Aliotta et. al., (2010), Exp Hematol., 38: 233-245). Previous studies have demonstrated that exosomes can horizontally transfer mRNAs to other cells, which can then be translated into functional proteins in the new location (Rana et al., (2012), supra; Hergenreider et. al., (2012), supra; Skog et al., (2008), supra). Similarly, miRNAs can be transferred by an exosomal route and further exert gene silencing in the recipient cells (Rana et al., (2012), supra; Aliotta et. al., (2010), supra; Katakowski et al., (2010), Cancer Res., 70: 8259-8263; Kosaka et al., (2010), J Biol Chem., 285: 17442-17452; Mittelbrunn et al., (2011), Nat Commun., 2: 282; Vogel et al., (2011), Anal Chem., 83: 3499-3506; Lee et al., (2012), Hum Mol Genet. 21: R125-134).

Since their discovery, a growing number of therapeutic applications are in development using exosomes derived from various producing cells, such as dendritic cells (DC), T lymphocytes, tumor cells and cell lines (Thery et al., (2002), Nat. Rev. Immunol. 2(8), 569-579; Delcayre et al., (2005), Expert Rev. Anti-Cancer Therapy 5(3), 537-547).

Exosomes, notably dendritic cell-derived exosomes (sometimes called dexosomes), have drawn considerable interest because of their immunological properties (Zitvogel et al. (1998), Nat Med., 4(5): 594-600; Thery et al., (1999), J. Cell Biol., 147(3): 599-610; Thery et al., (2002), Nat. Rev. Immunol., 2(8):569-579; Lamparski et al., (2002), J. Immunol. Methods., 270(2): 211-226; Vincent-Scheinder et al., (2002), Int. Immunol. 14(7): 713-722; Andre et al., (2004), J. Immunol. 172(4): 2126-2136. Their studies culminated with the evaluation of patient-derived dexosomes for the treatment of cancer (Delcayre et al., (2005), Expert Rev. Anticancer Ther. 5(3): 537-547). Two Phase I clinical trials of autologous dexosomes therapy for non-small cell lung (NSCL) and melanoma cancer patients, respectively, were completed that established the feasibility and safety of this approach (Morse et al., (2005), J. Transl. Med., 3(1):9; Escudier et al., (2005), J. Transl. Med., 3(1):10).

Exosomes derived from tumor cells, cell lines, T cells, are also being assessed as an alternative to dexosomes for the preparation of cancer vaccines (Wolfers et al., (2001), Nat. Med., 7(3): 297-303; Andre et al., (2002), Vaccine, 20(Suppl 4): A28-31; Andre et al., (2002), Lancet, 360(9329): 295-305; Altieri et al., (2004), J. Immunother., 27(4): 282-288).

Thus, as new exosome properties and technologies unveil, a growing number of possible applications are emerging in the fields of vaccine (Delcayre & Le Pecq, (2006), Curr. Opin. Mol. Ther., 8(1): 31-38), autoimmune diseases (Abusamra et al., (2005), Blood Cells Mol. Dis., 35(2): 169-173; Kim et al., (2005), J. Immunol. 174(10): 6440-6448; Taylor et al., (2006), J. Immunol., 176(3): 1534-1542 and transplantation (Morelli (2006), Am. J. Transplant. 6(2): 254-261; Peche et al., (2006), Am J. Transplant. 6(7): 1541-1550.

New studies are also showing that exosomes may play a critical role in some pathophysiological situations and therefore these vesicles are now also emerging as potential drug targets.

Exosomes can also be tailored to display a broad range of drug targets, including G protein-coupled receptors. Such vesicles provide a new source of complex membrane proteins that are maintained in their native conformation. Given the difficulties to isolate receptors for drug target validation and discovery, receptor presentation on exosome emerges as a promising new tool for drug screening.

Consequently, there is a continuing need for means of producing and/or isolating exosomes of different types from various cells for clinical, research and drug screening applications.

### SUMMARY

The inventors have discovered that exosomes can be produced from different cell types by exposing the cells to light between 500 nm to 820 nm. They have further identified that different types of exosomes can be produced by the same cell type simply by altering the wavelength of light that the cell is exposed to.

Accordingly, in a first aspect the present invention provides a method of producing exosomes or extracts thereof comprising the steps of:
(a) exposing a population of isolated mammalian cells to light between 500 nm to 820 nm for sufficient time to enable said cells to produce and excrete said exosomes; and
(b) separating said exosomes from other cellular components based on molecular weight, size, shape, composition or biological activity.

In some embodiments, the wavelength of the light is between 595-660 nm (10-80 mW), and/or 800-820 nm (30-120 mW) and/or 510-540 nm (10-100 mW) for at least 1 mins. In other embodiments, the wavelength of the light is 532 nm (10 mW), and/or 595 nm (30 mW) and/or 660 nm (30 mW) and/or 810 nm 100 mW) for at least 5 mins.

It will be appreciated by those skilled in the art that any mammalian cell that is capable of producing exosomes might be used in the methods of the present invention. In some embodiments, the isolated mammalian cells are hematopoietic cells, reticulocytes, monocyte-derived dendritic cells (MDDCs), monocytes, B lymphocytes, antigen-presenting cells, mastocytes or mesenchymal stem cells.

In a second aspect, the present invention provides exosomes or extracts thereof produced by a method according to the first aspect.

It is well documented that exosomes have a number of proposed uses including treating or preventing diseases or disorders that afflict mammals.

Accordingly, in a third aspect, the present invention provides a method of treating a patient using exosomes or extracts thereof comprising:
(i) isolating a population of cells from the patient;
(ii) exposing said population of cells to light between 500 nm and 820 nm for sufficient time to enable said cells to produce and excrete exosomes; and
(iii) administering the exosomes to the patient.

In a fourth aspect, the present invention provides a method for inducing an anti-tumor and anti-cachexia immune response in a mammal comprising the step of administering an exosome according to the second aspect.

In a fifth aspect, the present invention provides a method of inhibiting an autoimmune response in a subject in need of such treatment, comprising administering, to the subject, an effective amount of an exosome according to the second aspect.

In some embodiments the autoimmune response is manifested as an autoimmune disease selected from the group consisting of rheumatoid arthritis, juvenile rheumatoid arthritis, systemic lupus erythematoisis, scleroderma, Sjogren's syndrome, diabetes mellitus type I, Wegener's granulomatosis, multiple sclerosis, Crohn's disease, psoriasis, Graves' disease, celiac sprue, alopecia areata, central nervous system vasculitis, Hashimoto's thyroiditis, myasthenia gravis, Goodpasture's syndrome, autoimmune hemolytic anemia, Guillan-Barre syndrome, polyarteritis nodosa, idiopathic thrombocytic purpura, giant cell arteritis, primary biliary cirrhosis, Addison's disease, ankylosing spondylitis, Reiter's syndrome, Takayazu's arteritis, and vitiligo.

In a sixth aspect, the present invention provides a diagnostic kit, comprising at least one exosome according to the second aspect and instructions for use.

In a seventh aspect, the present invention provides a method of screening for active substances for the treatment or prevention of a disease or disorder comprising:
(i) providing at least one exosome according to the second aspect;
(ii) contacting a test substance with said exosome; and
(iii) comparing said exosome after being contacted with the test substance with a sample of exosome before being contacted with said test substance.

In an eighth aspect, the present invention provides a method of transferring genetic material to a cell comprising:
(a) providing at least one exosome according to the second aspect;
(b) transforming or transfecting said exosome with selected genetic material; and
(c) transferring the selected genetic material from the exosome to recipient cells.

In a ninth aspect, the present invention provides a method for diagnosing a disease or disorder in a subject, comprising:
(a) providing a biological sample from a subject;
(b) contacting said biological sample with at least one exosome according to the second aspect; and
(c) identifying whether said subject has said disease or disorder.

In a tenth aspect, the present invention provides a composition comprising at least one exosome according to the second aspect and a pharmaceutically acceptable carrier.

### BRIEF DESCRIPTION OF THE FIGURES

FIG. 1 shows the counts of particle dynamics in the filtrate of tissue culture medium obtained from cultures of about 7.2 million monocytes in RPMI following exposure to laser light for 5 and 10 min, red and yellow light (10 min), and green light (5 min and 10 min) using Izon's qNano Technology.

FIG. 2 shows the baseline duration of filtrate of tissue culture medium obtained from cultures of about 7.2 million monocytes in RPMI following exposure to laser light for 5 and 10 min, red and yellow light (10 min), and green light (5 min and 10 min) using Izon's qNano Technology.

### DESCRIPTION OF THE PREFERRED EMBODIMENTS OF THE INVENTION

It is to be understood that this disclosure is not limited to particularly exemplified methods and may, of course, vary. It is also to be understood that the terminology used herein is for the purpose of describing particular embodiments only, and is not intended to be limiting which will be limited only by the appended claims.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the disclosed methods. Nothing herein is to be construed as an admission that what is disclosed herein is not entitled to antedate such disclosure by virtue of prior invention.

In this specification and in the claims that follow, reference will be made to a number of terms that shall be defined to have the following meanings:

The term “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

It must be noted that, as used in the specification and the appended claims, the singular forms “a,” “an” and “the” include plural referents unless the context clearly dictates otherwise. Thus, for example, reference to “an exosome” includes mixtures of two or more exosomes, and the like.

The present invention may be practiced in conjunction with various cell or tissue separation techniques that are conventionally used in the art, and only so much of the commonly practiced process steps are included herein as are necessary to provide an understanding of the present invention.

The inventors have determined that it is surprisingly advantageous to irradiate isolated mammalian cells with one or more specific wavelengths of light, especially yellow, red and/or green wavelengths of light or combinations thereof or infrared light, in order to produce, increase or alter the expression/production of exosomes.

It will be appreciated by those skilled in the art that visible light comprises different colour light having different frequency or wavelength. Table 1 shows the various colours of the visible light spectrum, while Table 2 shows the colour, wavelength, frequency and energy of light.

TABLE 1

| Colour | wavelength interval | frequency interval |
|---|---|---|
| Red | ~700-635 nm | ~430-480 THz |
| Orange | ~635-590 nm | ~480-510 THz |
| Yellow | ~590-560 nm | ~510-540 THz |
| Green | ~560-490 nm | ~540-610 THz |
| Blue | ~490-450 nm | ~610-670 THz |
| Purple | ~450-400 nm | ~670-750 THz |

TABLE 2

| Colour | λ (nm) | ν (THz) | ν_{b} (μm⁻¹) | E (eV) | E (kJ mol⁻¹) |
|---|---|---|---|---|---|
| Infrared | >1000 | <300 | <1.00 | <1.24 | <120 |
| Red | 700 | 428 | 1.43 | 1.77 | 171 |
| Orange | 620 | 484 | 1.61 | 2.00 | 193 |
| Yellow | 580 | 517 | 1.72 | 2.14 | 206 |
| Green | 530 | 566 | 1.89 | 2.34 | 226 |
| Blue | 470 | 638 | 2.13 | 2.64 | 254 |
| Purple | 420 | 714 | 2.38 | 2.95 | 285 |
| Near ultraviolet | 300 | 1000 | 3.33 | 4.15 | 400 |
| Far ultraviolet | <200 | >1500 | >5.00 | >6.20 | >598 |

Preferably, one or more lasers can be used as a source of the light. While yellow light, red light and green light or combinations thereof are preferred all lights sources may be used in the claimed method.

By “yellow”, “red” and “green” light, we include those wavelengths of light associated with those particular colours. However, preferably in the method of the first aspect of the invention the following wavelengths of light and power rating are used: 575-595 nm (5-20 mW) (yellow; this can also be considered to be an “orange” range of wavelengths as well), and 630-635 nm or 660-670 nm (10-100 mW) (red) and/or 510-540 nm (10-60 mW) (green) for 30-60 mins. An embodiment of this aspect of the invention is wherein the cells are irradiated with 595 nm (20 mW), 635 nm (60 mW) and 535 nm (60 mW), of light for at least 5 mins.

Methods of isolating and preparing population of cells that can be used in the method of the invention will vary according to the cell type to be used, and tissue they are to be isolated from. Many examples of methods for preparing cells from particular tissues are known and the skilled person would be able to use such methods when preparing a population to be used.

For example, with regard to bone marrow (mesenchymal) stem cells there are many laboratory methods well known in the art that can be used directly or readily adapted so as to provide a population of such stem cells for the invention. Similarly, there are many protocols well known in the art that can be used to isolate peripheral blood cells for the invention.

In the preferred embodiment, the sources of light could be derived from a light emitting diode, a laser, a fluorescent light source, an organic light emitting diode, a light emitting polymer, a xenon arc lamp, a metal halide lamp, a filamentous light source, an intense pulsed light source, a sulphur lamp, and combinations thereof. The preferred embodiment would be to irradiate the cell population with a combination of laser diodes emitting light wavelengths and power ratings: 575-595 nm (5-20 mW) (yellow; this can also be considered to be an “orange” range of wavelengths as well), and 630-635 nm or 660-670 nm (10-100 mW) (red) and/or 510-540 nm (10-60 mW) (green) for at least 5 mins, where the sample is placed at a distance of 0-30 cm. More preferably the cells are irradiated with 595 nm (20 mW), 635 nm (60 mW) and 535 nm (60 mW), of light for at least 5 mins.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance can or cannot occur, and that the description includes instances where the event or circumstance occurs and instances where it does not.

An “exosome” refers to cell-derived vesicle that is prepared as described herein; however, exosomes are naturally present in many and perhaps all biological fluids, including blood, urine, and synovial fluid. The reported diameter of exosomes is between 30 and 150 nm. The exosomes of the present invention can be used to treat, diagnose, cure, mitigate, prevent (i.e., prophylactically), ameliorate, modulate, or have an otherwise favourable effect on a disease, disorder, infection, and the like.

The exosomes of the present invention may be administered topically, enterally or parenterally. For example, the exosomes can be administered orally, nasally, intravenously, intramuscularly, subcutaneously, sublingually, intrathecally, intraperitoneally, intratumorally, topically, transdermally or intradermally. The route of administration can depend on a variety of factors, such as the environment and therapeutic goals. Further non-limiting pharmaceutically suitable materials that may be incorporated in pharmaceutical preparations/compositions disclosed herein include absorption enhancers, pH-adjusting agents and buffers, osmolarity adjusters, preservatives, stabilizers, antioxidants, surfactants, thickening agents, co-solvents, emollients, dispersing agents, flavouring agents, colouring agents and wetting agents and ligands/pilote/targeting molecules. The exosomes may be in the form of a liquid, a powder, an aerosol, a capsule, a tablet, a suppository, a cream, a gel and an ointment. Exemplary types of liquid include a lotion and a spray. Methods for preparing appropriate formulations are well known in the art (see, for example, Hendrickson, R. Ed. Remington: The Science and Practice of Pharmacy, 21^{st} ed.; Lippincott Williams & Wilkins: Baltimore Md., 2005).

The disclosure will now be further described by way of reference only to the following non-limiting examples. It should be understood, however, that the examples following are illustrative only, and should not be taken in any way as a restriction on the generality of the disclosure described above.

### Example 1

### Effect of Light on Human Peripheral Blood Monocytes Microparticle Secretion and Dynamics

In this experiment the inventor used different frequencies of light and different exposure times to ascertain if these had differing effects on exosome secretion from fresh human peripheral blood monocytes.

Blood was collected from a healthy male volunteer by venipuncture and placed in sodium citrate (anti-coagulant). Monocytes were then isolated from cell suspensions by size sedimentation using a continuous gradient of colloidal silica particles (Percoll™) by standard techniques.
Percoll (specific gravity 1.130 g/ml, 17 mOs/kg water; Amersham Biosciences)
2× phosphate-buffered saline (PBS; Invitrogen)
15-ml conical polypropylene centrifuge tubes
Sorvall RC2-B centrifuge with SS-34 fixed-angle rotor (or equivalent)
15-ml polycarbonate centrifuge tubes (Sorvall)
Beckman GPR centrifuge with GH-3.7 horizontal rotor (or equivalent temperature-controlled centrifuge)
Additional reagents and equipment for cell counting.

All solutions and equipment coming into contact with cells were sterile, and aseptic technique was used accordingly.

The methodology used was as follows:

1. Whole blood was centrifuged for 15 min at 200×g at room temperature and the supernatant, which contained platelets, was discarded. The blood was then resuspended at a cell density 2-5×10⁷ cells per 1 to 2 ml of suspension in PBS.

2. A continuous gradient was then formed in a 15-ml polycarbonate centrifuge tube by mixing 7 ml of Percoll and 6 ml of 2×PBS and then centrifuging for 40 min at 21,000×g at room temperature.

3. The blood from step 1 was then gently layered onto the preformed gradient.

4. The material was centrifuged for 20 min at 1000×g at room temperature.

5. Using sterile Pasteur pipets the four opaque bands of cells beginning at the top was carefully collected. Band 1 contained dead cells, debris, and a few platelets. Band 2 contained monocytes, a few lymphocytes, and any remaining platelets. Band 3 contained lymphocytes and a few monocytes. Band 4 contained granulocytes and red blood cells.

6. The cells in band 2, which comprised 70% to 90% monocytes, were washed and counted and 7.2 million monocytes where then plated in RPMI media into each well of a six well plate. Laser and LED probes where positioned 1 cm away from the bottom of the plate and the cells were exposed to light.

8. Culture medium was then removed and filtered through a 220 nm cut-off filter (Milipore).

9. The filtrate was then counted for particle dynamics using Izon's qNano Technology (www.Izon.com). The Izon Nanopore filter used for measuring was the NP200, which measures particles between 100 to 400 nanometres in diameter.

Izon's qNano technology was employed to detect the size of particles in 100,000 g pellets from ECC-1 cell culture medium, uterine fluid and mucus. The detector records the particle blockade rate while the pressure applied, across a pore sensor is varied. In practice it enables accurate particle-by-particle characterization of vesicles from 50 nm to greater than 1 μm in size in complex mixtures, without averaging the particle sizes.

Representative particle diameters from culture media (passed through a 220 nanometer filter prior to analysis) were analysed. Exosomes are 30-150 nm, microvesicles are 100 nm-1 μm and apoptotic bodies are 500 nm-3 μm in diameter.

While exosomes are now being widely studied in a variety of systems, particularly in relation to cancer, the particle size in their preparations is often not adequately defined. Apoptotic bodies, which are generally of 500-3000 nm are a common contaminant and can provide misleading data. The 100,000 g fractions in this study were measured using qNano technology which measures every particle individually and which defined a major peak in each preparation between 50-150 nm, the size of exosomes, but with overlap with microvesicles from 100-1000 nm, By passing the sample through a Millipore filter (with a 220 nm cut off we expected that most of the population of vesicles would be exosomes.

As expected the tail from these peaks extended to 220 nm, but decreased thereby suggesting larger microvesicles were also present but in much smaller amounts

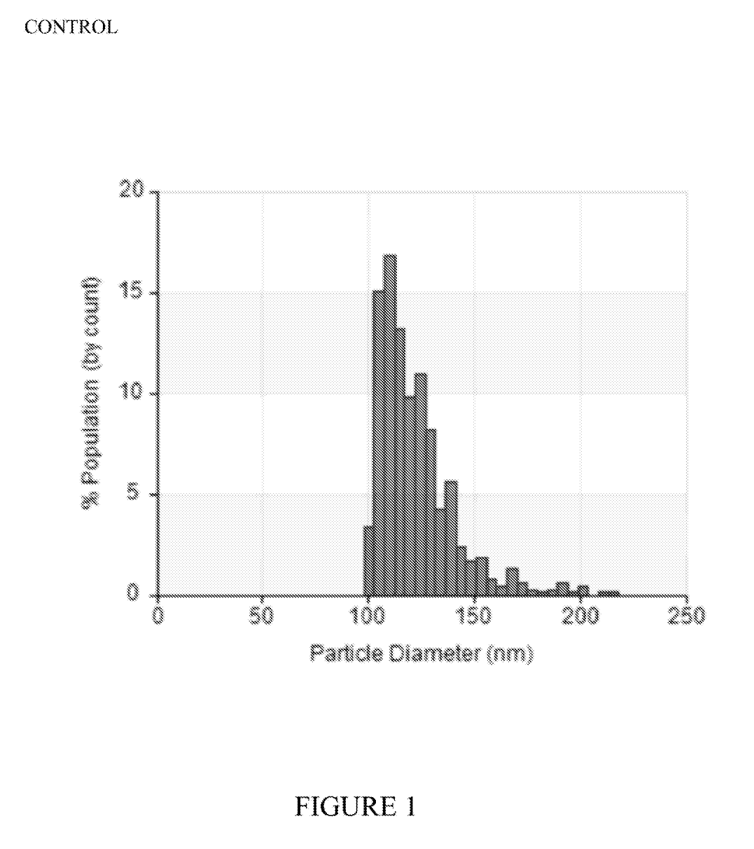

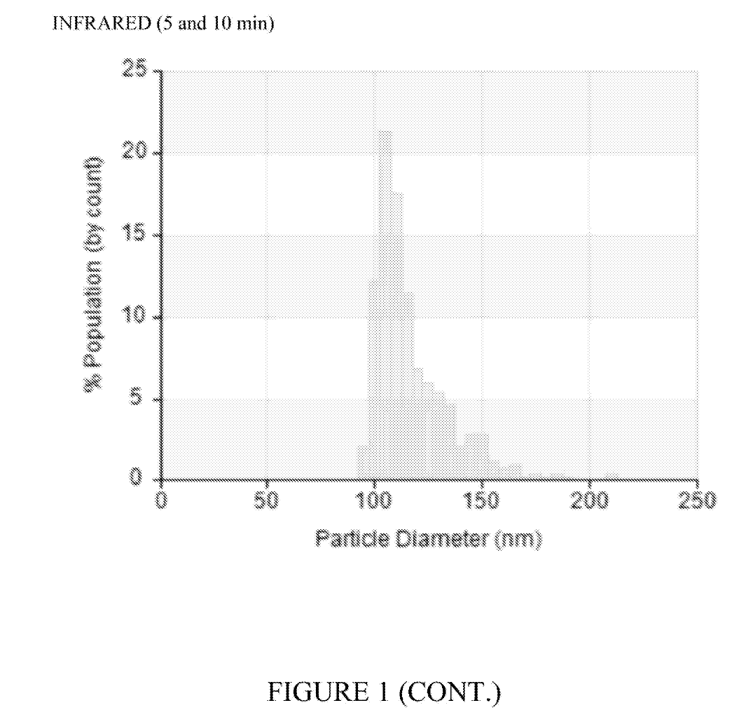

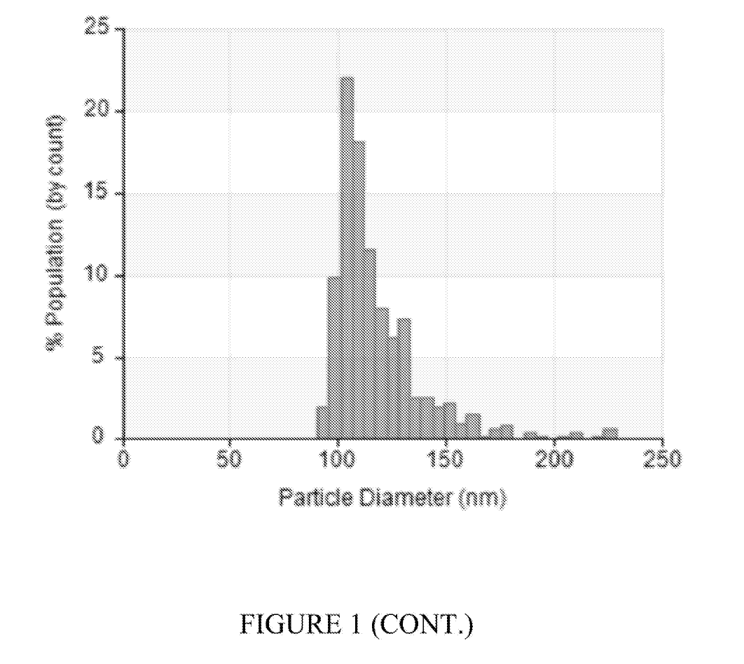

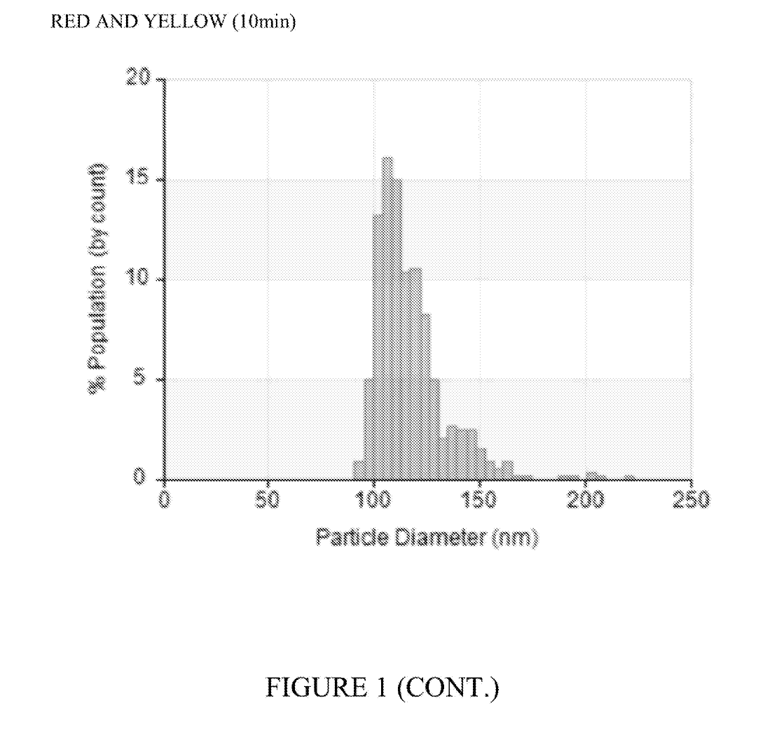

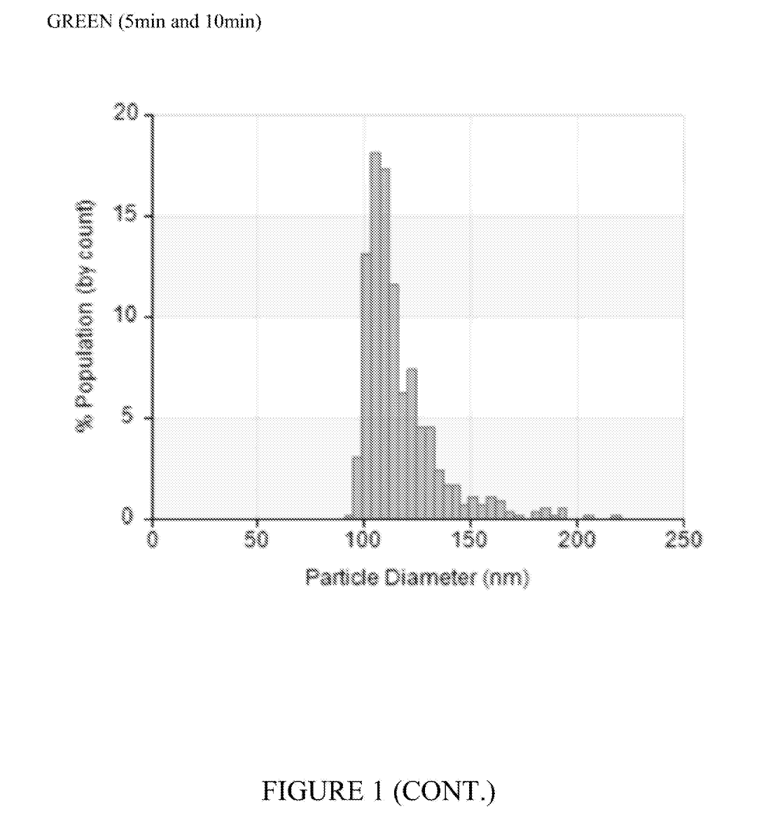

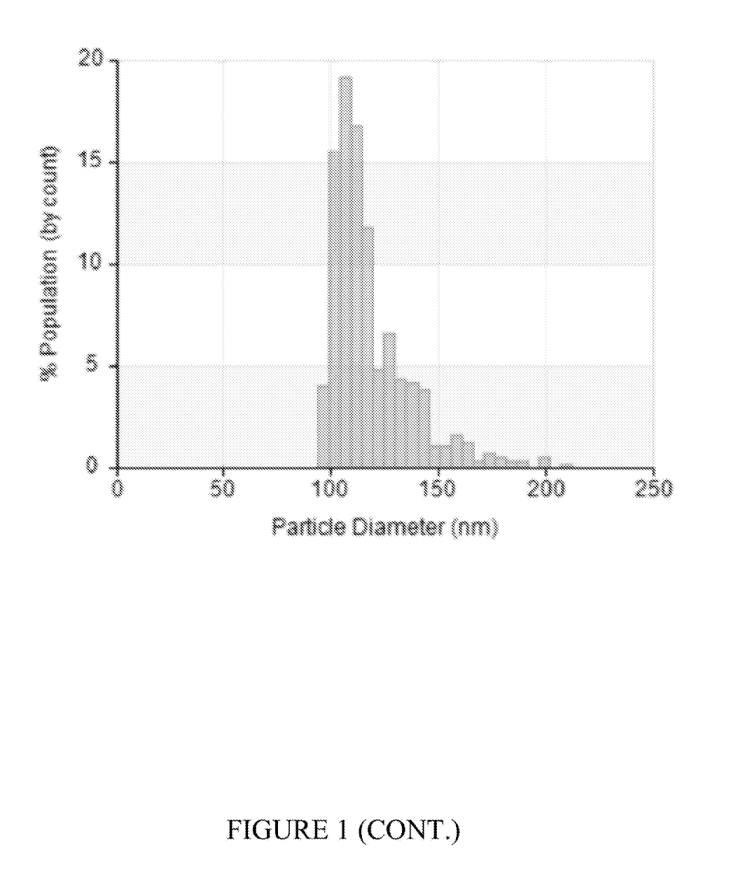

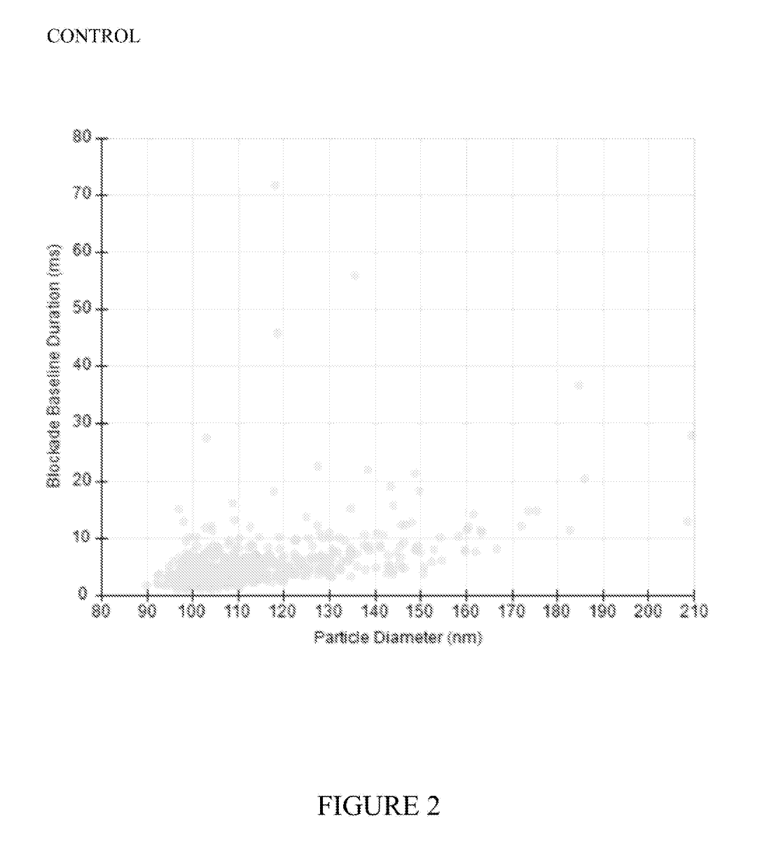

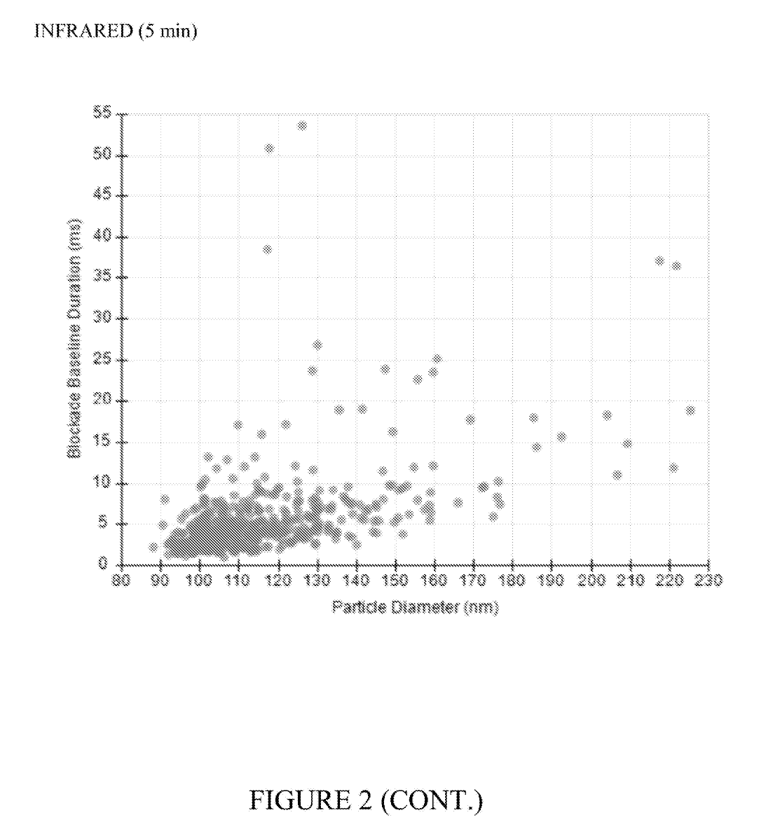

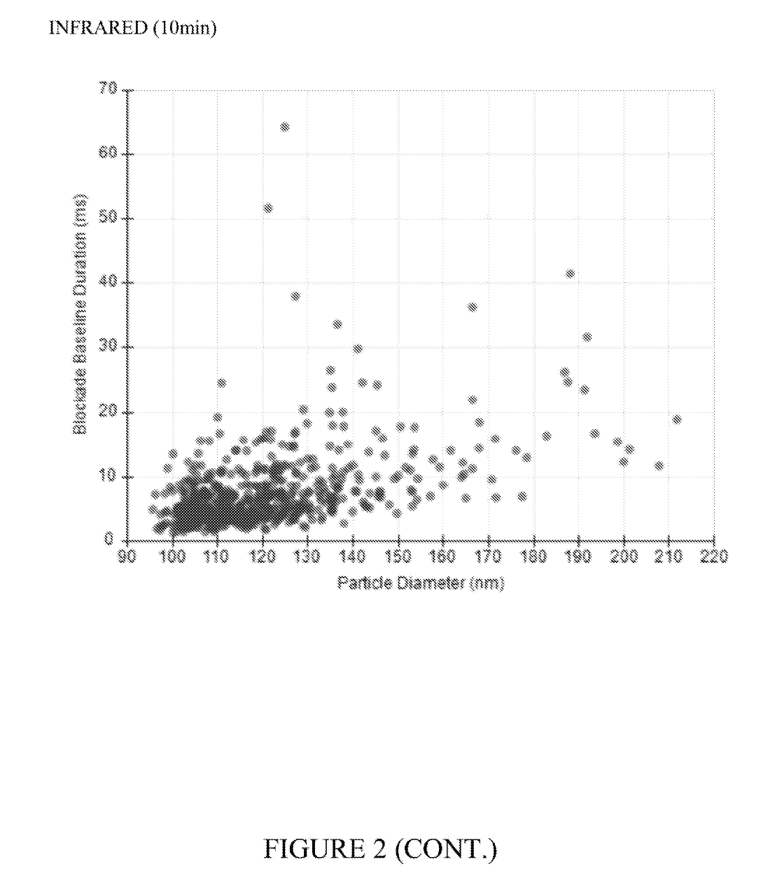

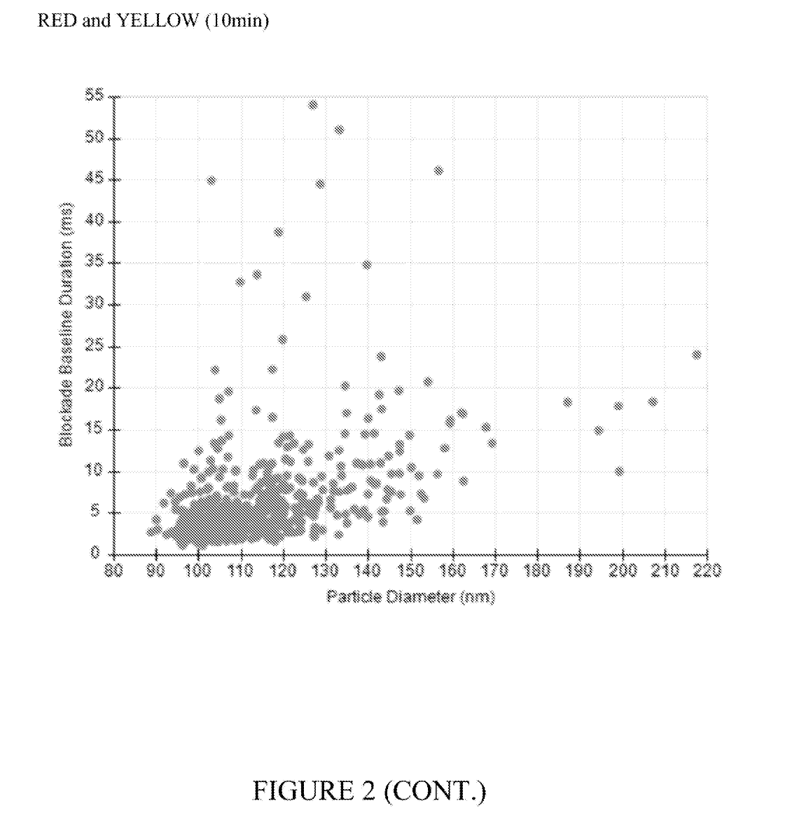

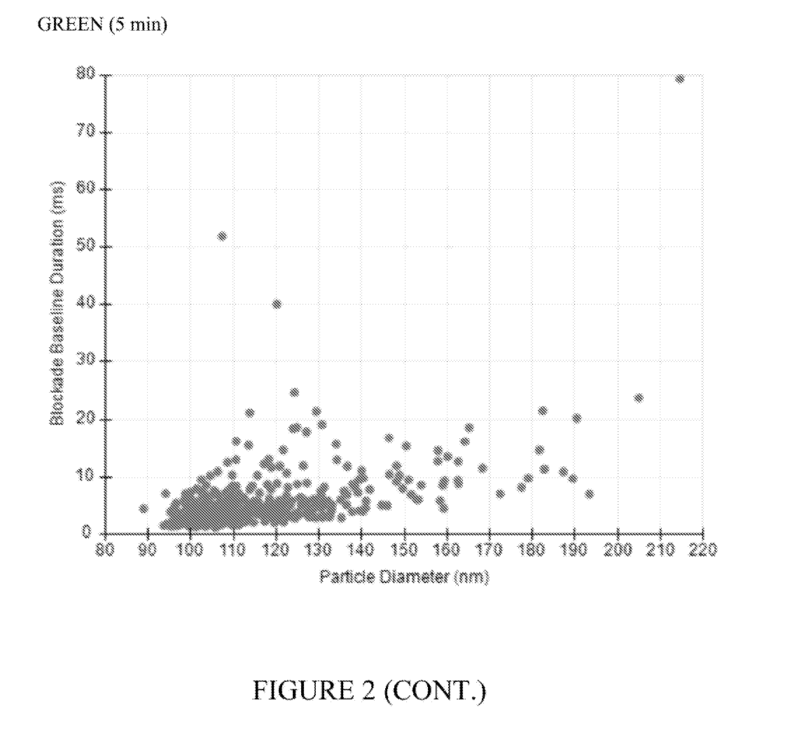

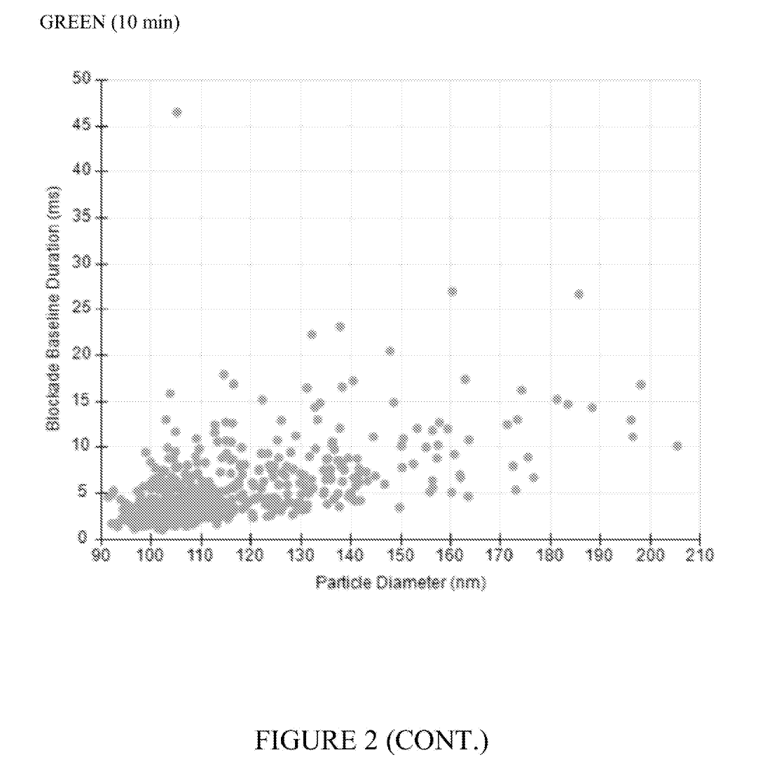

Table 3 and FIGS. 1 and 2 show the effect of different wavelengths of light on the cells prepared above.

TABLE 3

| | | | | | | |
|---|---|---|---|---|---|---|
| Wavelength of light (nm) | None | 810 | 810 | 660 + 595 | 532 | 532 |
| Colour | None | Infrared | Infrared | red + yellow | Green | Green |
| Source of light | None | Laser | Laser | LED | Laser | Laser |
| Intensity of light (mW) | None | 100 | 30 | 10 | 10 | 10 |
| Exposure time (min) | None | 5 | 10 | 10 | 5 | 10 |
| Particle rate (particles/min) | 106 | 109 | 115 | 120 | 143 | 168 |
| Mean concentration (particles/ml) | 41.3 mil | 42.2 mil | 45.0 mil | 47.2 mil | 56.2 mil | 65.8 mil |
| Average particle diameter (nm) | 120 | 115 | 116 | 115 | 115 | 116 |
| Min particle diameter (nm) | 96 | 90 | 88 | 89 | 89 | 91 |
| Max Particle diameter (nm) | 212 | 209 | 225 | 218 | 215 | 205 |
| Most common particle diameter (nm) | 108 | 102 | 101 | 104 | 104 | 104 |
| Average baseline duration (ms) | 7.47 | 5.76 | 5.57 | 6.65 | 5.3 | 5.47 |
| Min baseline duration (ms) | 1.26 | 1.16 | 0.94 | 1.12 | 1.1 | 1.06 |
| Max baseline duration (ms) | 64.36 | 71.78 | 53.66 | 54.1 | 79.4 | 46.52 |
| Most common baseline duration (ms) | 4.28 | 3.51 | 3.99 | 2.78 | 3.58 | 3.32 |
| Average FWHM Duration (ms) | 1.16 | 0.87 | 0.82 | 1.01 | 0.82 | 0.8 |
| Min FWHM Duration (ms) | 0.38 | 0.23 | 0.33 | 0.23 | 0.16 | 0.23 |
| Max FWHM Duration (ms) | 9.95 | 10.7 | 13.12 | 10.29 | 8.19 | 5.62 |
| Most common FWHM Duration (ms) | 0.92 | 0.68 | 0.68 | 0.64 | 0.71 | 0.58 |

The conclusions that are drawn from the above data are that as the wavelength of light decreased the particle number increased. In other words, light exposure stimulated exosome secretion, which is increased as the wavelength of light decreased.

Doubling the exposure time increased the particle count, which means that increased exposure to light stimulated microparticle secretion.

All light exposures decreased the average particle diameter, which means light exposure decreased the overall diameter of microparticles secreted. Alternatively, that light exposure stimulated the release of smaller microparticles or that light exposure stimulated the release of exosomes only and not microvesicles.

We found that infrared and green laser light decreased the baseline duration. We interpreted this to mean that infrared and green laser light causes an increase in the surface charge of the microparticles secreted. However, it could also mean that more exosomes where secreted which have a higher surface charge than microvesicles.

We also observed that infrared and green laser light decreased the full width at half maximum (FWHM) duration, while red and yellow LED decreased slightly the FWHM duration. We interpreted this to mean that the nature of the 3D shape (speed and length) of the microparticles changed with light exposure suggests that light exposure caused the secretion of a particular type of microparticle (exosome). Cells exposed to red and/or yellow LED exposure secretes a different type of exosome compared to infrared and green laser light.

1. A method of producing exosomes or extracts thereof comprising the steps of:
(a) isolating a population of mammalian cells;
(b) exposing said cells to light between 500 nm to 820 nm for at least 5 mins to enable said cells to produce and excrete said exosomes; and
(c) separating said exosomes from other cellular components based on molecular weight, size, shape, composition or biological activity.

2. The method of claim 1, wherein the wavelength of the light is between 595-660 nm (10-80 mW), 800-820 nm (30-120 mW) or 510-540 nm (10-100 mW) or combinations thereof.

3. The method of claim 1, wherein the wavelength of the light is 532 nm (10 mW), 595 nm (30 mW), 660 nm (30 mW) or 810 nm (100 mW) or combinations thereof.

4. (canceled)

5. The method of claim 1, wherein the time period is at least 15 mins.

6. The method of claim 1, wherein the population of isolated mammalian cells are selected from the group consisting of hematopoietic cells, reticulocytes, monocyte-derived dendritic cells (MDDCs), monocytes, B lymphocytes, antigen-presenting cells, mastocytes and mesenchymal stem cells.

7. The method of claim 5, wherein the population of isolated mammalian cells is monocytes.

8. Exosomes or extracts thereof produced by the method of claim 1.

9. A composition for use in the treatment of patient comprising exosomes or extracts thereof produced by the method of claim 1.

10. A method of treating a patient in need thereof using exosomes or extracts thereof comprising:
(i) isolating a population of cells from the patient;
(ii) exposing said population of cells to light between 500 nm and 820 nm for
at least 5 mins to enable said cells to produce and excrete exosomes; and
(iii) administering the exosomes to the patient.

11. The method of claim 9, wherein the wavelength of the light is between 595-660 nm (10-80 mW), 800-820 nm (30-120 mW) and 510-540 nm (10-100 mW) or combinations thereof.

12. The method of claim 9, wherein the wavelength of the light is 532 nm (10 mW), 595 nm (30 mW), 660 nm (30 mW) or 810 nm (100 mW) or combinations thereof.

13. (canceled)

14. The method of claim 9, wherein the time period is at least 15 mins.

15. The method of claim 9, wherein the population of isolated mammalian cells are selected from the group consisting of hematopoietic cells, reticulocytes, monocyte-derived dendritic cells (MDDCs), monocytes, B lymphocytes, antigen-presenting cells, mastocytes and mesenchymal stem cells.

16. The method of claim 9, wherein the population of isolated mammalian cells is monocytes.

17. The method of claim 9, wherein the exosomes are administered orally, nasally, intravenously, intramuscularly, subcutaneously, sublingually, intrathecally, intraperitoneally, intratumorally, topically, transdermally and intradermally.

18. A method for inducing an anti-tumor and anti-cachexia immune response in a mammal comprising the steps of
(i) isolating a population of cells from the mammal;
(ii) exposing said population of cells to light between 500 nm and 820 nm for
at least 5 mins to enable said cells to produce and excrete exosomes; and
(iii) administering the exosomes to the mammal.

19. The method of claim 16, wherein the exosomes are administered orally, nasally, intravenously, intramuscularly, subcutaneously, sublingually, intrathecally, intraperitoneally, intratumorally, topically, transdermally and intradermally.

20. A method of inhibiting an autoimmune response in a subject in need of such treatment, comprising the steps of
(i) isolating a population of cells from the subject;
(ii) exposing said population of cells to light between 500 nm and 820 nm for
at least 5 mins to enable said cells to produce and excrete exosomes; and
(iii) administering the exosomes to the subject.

21. A method according to claim 18, wherein the autoimmune response is manifested as an autoimmune disease selected from the group consisting of rheumatoid arthritis, juvenile rheumatoid arthritis, systemic lupus erythematoisis, scleroderma, Sjogren's syndrome, diabetes mellitus type I, Wegener's granulomatosis, multiple sclerosis, Crohn's disease, psoriasis, Graves' disease, celiac sprue, alopecia areata, central nervous system vasculitis, Hashimoto's thyroiditis, myasthenia gravis, Goodpasture's syndrome, autoimmune hemolytic anemia, Guillan-Barre syndrome, polyarteritis nodosa, idiopathic thrombocytic purpura, giant cell arteritis, primary biliary cirrhosis, Addison's disease, ankylosing spondylitis, Reiter's syndrome, Takayazu's arteritis, and vitiligo.

22. A diagnostic kit, comprising at least one exosome produced by the method of claim 1 and instructions for use.

23. A method of screening for active substances for the treatment or prevention of a disease or disorder comprising:
(i) providing at least one exosome produced by the method of claim 1;
(ii) contacting a test substance with said exosome; and
(iii) comparing said exosome after being contacted with the test substance with a sample of exosome before being contacted with said test substance.

24. A method of transferring genetic material to a cell comprising:
(a) providing at least one exosome produced by the method of claim 1,
(b) transforming or transfecting said exosome with selected genetic material; and
(c) transferring the selected genetic material from the exosome to recipient cells.

25. A composition comprising at least one exosome produced by the method of claim 1 and a pharmaceutically acceptable carrier.

26. (canceled)
